(19) United States
(12) Patent Application Publication
(10) Pub. No.: US 2024/0218436 A1
(43) Pub. Date: Jul. 4, 2024

(54) AMPLIFICATION TECHNIQUES FOR NUCLEIC ACID CHARACTERIZATION

(71) Applicant: ILLUMINA, INC., San Diego, CA (US)

(72) Inventors: Niall Anthony Gormley, Cambridge (GB); Clifford Lee Wang, Redwood City, CA (US)

(21) Appl. No.: 18/558,057

(22) PCT Filed: Apr. 28, 2022
(86) PCT No.: PCT/US2022/026777

§ 371 Date: Oct. 30, 2023

Related U.S. Application Data
(60) Provisional application No. 63/181,769, filed on Apr. 29, 2021.

(51) Int. Cl. C12Q 1/6844; C12Q 1/6816

(52) U.S. Cl. CPC C12Q 1/6844; C12Q 1/6816

(57) ABSTRACT

Nucleic acid amplification techniques are disclosed. Embodiments include generating concatenated nucleic acids using rolling circle amplification of templates, e.g., starting from a cDNA of a full-length mRNA or from synthetic templates, and sequencing and/or detecting the concatenated nucleic acids. In some embodiments, the technology disclosed includes amplification reactions that include CRISPR-Cas interactions that generate primers as a result of the CRISPR-Cas interactions, whereby primers are in turn used as part of detectable amplification reactions. The disclosed amplification techniques may use synthetic oligonucleotides or primers.

### BACKGROUND

The disclosed technology relates generally to nucleic acid characterization, e.g., detection and/or sequencing techniques. In some embodiments, the technology disclosed includes generating concatenated nucleic acids using rolling circle amplification, e.g., starting from a cDNA of a full-length mRNA or from synthetic templates, and sequencing and/or detecting the concatenated nucleic acids. In some embodiments, the technology disclosed includes amplification reactions that include CRISPR-Cas interactions that generate primers as a result of the CRISPR-Cas interactions, whereby primers are in turn used as part of detectable amplification reactions. The disclosed amplification techniques may use synthetic oligonucleotides or primers.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Advances in the study of biological molecules have been led, in part, by improvement in technologies used to characterize the molecules or their biological reactions. In particular, the study of the nucleic acids DNA and RNA has benefited from developing technologies used for sequence analysis. Methods for sequencing a polynucleotide template can involve performing multiple extension reactions using a DNA polymerase or DNA ligase, respectively, to successively incorporate labelled nucleotides or polynucleotides complementary to a template strand. In such sequencing-by-synthesis reactions, a new nucleotide strand base-paired to the template strand is built up by successive incorporation of nucleotides complementary to the template strand. In certain circumstances the amount of sequence data that can be reliably obtained with the use of sequencing-by-synthesis techniques may be limited. In some circumstances the sequencing run may be limited to a number of bases that permits sequence realignment, for example around 25-30 cycles of incorporation. However, for applications such as, for example, SNP analysis, variant analysis, and haplotyping, it would be advantageous in many circumstances to be able to reliably obtain further sequence data for the same template molecule. Further, when the starting material used in the sequencing reaction is of low concentration, the sequencing data from 25-30 cycles may be insufficient for the desired analysis. Thus, there exists a need for new methods that facilitate the targeted next generation sequencing for low concentration starting material and/or that can sequence or detect SNPs or other variant sequences, e.g., somatic mutations, viral variants.

### BRIEF DESCRIPTION

In one embodiment, the present disclosure provides a nucleic acid composition. The nucleic acid composition includes a first oligonucleotide comprising a first 5′ primer sequence, a first 3′ primer sequence, and a first intervening region disposed between the first 5′ primer sequence and the first 3′ primer sequence and a second oligonucleotide comprising a second 5′ primer sequence, a second 3′ primer sequence and a second intervening region disposed between the second 5′ primer sequence and the second 3′ primer sequence. The nucleic acid composition also includes a target nucleic acid, wherein the first 5′ primer sequence and the first 3′ primer sequence are complementary to first regions flanking a first target sequence of the target nucleic acid and wherein the second 5′ primer sequence and the second 3′ primer sequence are complementary to second regions flanking a second target sequence of the target nucleic acid such that the first oligonucleotide, when bound to the target nucleic acid, forms a first looped structure about the first target sequence and the second oligonucleotide, when bound to the target nucleic acid, forms a second looped structure around the second target sequence.

In one embodiment, the present disclosure provides a method for amplifying a target sequence including steps of contacting a target nucleic acid with an oligonucleotide such that the oligonucleotide binds to spaced-apart target binding sequences on the nucleic acid to form a looped structure about a target sequence of the target nucleic acid; extending a 3′ end of the oligonucleotide towards a 5′ end and across the target sequence; ligating the extended 3′ end to the 5′ end of the oligonucelotide to form a closed loop; and using the closed loop as a template for rolling circle amplification to generate a concatenated single-stranded nucleic acid.

In one embodiment, the present disclosure provides a method for detecting a target nucleic acid including steps of providing a system having a first clustered regularly interspaced short palindromic repeats (CRISPR) guide RNA and a first CRISPR-associated (Cas) protein and a second CRISPR guide RNA and a second Cas protein, wherein the first guide RNA contains a target-specific nucleotide region complementary to a first region of a target nucleic acid and the second guide RNA contains a target target-specific nucleotide region complementary to a second region of a target nucleic acid spaced apart from the first region; contacting the target nucleic acid with the system to form a complex to cleave within the first region and the second region to release an oligonucleotide comprising intervening nucleotides between the first region and the second region; annealing the oligonucleotide to a template; and amplifying the template using the annealed oligonucleotide as a primer.

In one embodiment, the present disclosure provides a method for detecting a target nucleic acid including steps of providing a system having a clustered regularly interspaced short palindromic repeats (CRISPR) guide RNA and a CRISPR-associated (Cas) protein, wherein the guide RNA contains a target-specific nucleotide region complementary to a region of a target nucleic acid; providing a plurality of circularized oligonucleotides; contacting the target nucleic acid with the system to form a complex; linearizing the plurality of circularized oligonucleotides to generate primers using the Cas protein in the complex; annealing one or more of the primers to a template; and amplifying the template using the one or more of the primers annealed to the template.

In one embodiment, the present disclosure provides a method for amplifying an mRNA target nucleic acid including steps of providing a primer for a reverse transcriptase reaction, the primer comprising a primer binding sequence for rolling circle amplification and a phosphorylated 5′ end; annealing the primer to an mRNA; extending the primer using reverse transcriptase to generate a cDNA comprising primer; ligating the phosphorylated 5′ end of the primer in the cDNA to a 3′ end to circularize the cDNA; annealing a rolling circle amplification primer to the primer binding sequence of the circularized cDNA; and amplifying the circularized cDNA using the rolling circle amplification primer annealed to the circularized cDNA to generate a concatenated single-stranded nucleic acid.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of oligonucleotides for use in rolling circle amplification-based characterization of nucleic acids, in accordance with embodiments of the present disclosure.

FIG. 2 is a schematic illustration of protocol steps in rolling circle amplification-based characterization of nucleic acids, in accordance with embodiments of the present disclosure.

FIG. 3 is a schematic illustration of a multiplexed rolling circle amplification-based characterization of nucleic acids, in accordance with embodiments of the present disclosure.

FIG. 4 is a schematic illustration of an example protocol starting from cDNA and that generates sense and antisense strand amplicons, in accordance with embodiments of the present disclosure.

FIG. 5 is a schematic illustration of an example protocol of steps for CRISPR-Cas mediated generation of a rolling circle amplification template for characterization of nucleic acids, in accordance with embodiments of the present disclosure.

FIG. 6 is a schematic illustration of an example protocol of steps for CRISPR-Cas mediated generation of a rolling circle amplification template for characterization of nucleic acids, in accordance with embodiments of the present disclosure.

FIG. 7 is a schematic illustration of an example protocol of steps for CRISPR-Cas mediated generation of a rolling circle amplification template for characterization of nucleic acids, in accordance with embodiments of the present disclosure.

FIG. 8 is a schematic illustration of a protocol for generation of double-stranded, concatenated full-length cDNA product by rolling circle amplification and second-strand synthesis;

FIG. 9 is a block diagram of a sequencing device configured to acquire sequencing data in accordance with the present techniques.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Described herein are a variety of methods and compositions that allow for the characterization of nucleic acids. Nucleic acid characterization may include acquiring sequence information and/or sequence detection data, such as data from polymerase chain reaction (PCR) and detection of amplicons, hybridization-based detection, array-based detection, etc. In certain embodiments, the disclosed techniques provide sequencing and/or detection techniques for target nucleic acids. In certain embodiments, provided herein are non-naturally occurring nucleic acids, e.g., recombinant and/or synthetic oligonucleotides, that are templates for amplification. In one embodiment, non-naturally occurring nucleic acid are used as templates to generate amplicons via rolling circle amplification. The generated amplicons may be provided for further processing in sequencing and/or detection protocols to generate sequencing and/or detection outputs.

Certain embodiments disclosed herein are discussed with reference to RNA target nucleic acids, e.g., single-stranded RNA. However, it should be understood that the embodiments may be used in conjunction with DNA or RNA that is single-stranded or double-stranded. In certain cases, double-stranded nucleic acids may be denatured as part of the disclosed protocols to generate single-stranded nucleic acid target nucleic acids where appropriate.

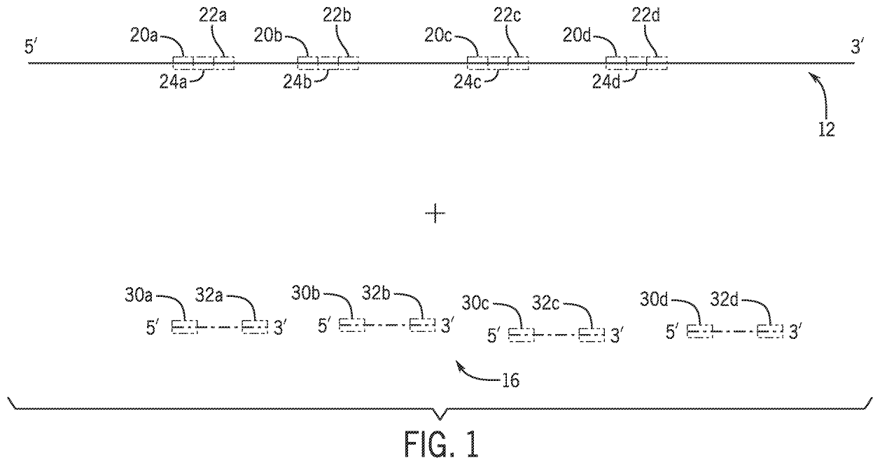

FIG. 1 shows a schematic illustration of components of a nucleic acid characterization technique using rolling circle amplification according to the disclosed embodiments. The technique includes a target nucleic acid 12, shown as single-stranded. The target nucleic acid may be a naturally-occurring single-stranded molecule, such as mRNA or a single-stranded virus, or may be denatured such that the target nucleic acid 12 also includes a reverse complement strand.

While the target nucleic acid 12 is shown for illustrative purposes as a single strand, it should be understood that the target nucleic acid may include multiple nucleic acid strands that may be the same or different from one another. For example, when the target nucleic acid 12 is a virus, the target nucleic acid 12 may include the virus genome on one nucleic acid strand (or a few strands). Multiple copies of the virus genome may be present that are generally a same sequence, although certain copies of the target nucleic acid 12 may have variation representative of sequence diversity within an infected individual. Further, where the sample is multiplexed, the target nucleic acid 12 may also include variation representative of inter-individual variation in virus sequence. Where the target nucleic acid 12 is a larger genome, the target nucleic acid 12 may be fragmented, with different strands representing different genome portions. Where the target nucleic acid 12 is a transcriptome, different strands represent different mRNA transcripts or cDNA copies thereof. In an embodiment, the target nucleic acid may be a viral nucleic acid, e.g., a COVID-19 RNA genome, from an infected individual, whereby the viral nucleic acid may include intra or inter-individual variants that can be detected, e.g., sequenced, by the disclosed techniques. In an embodiment, disclosed techniques are used as part of a multiplexed sample analysis.

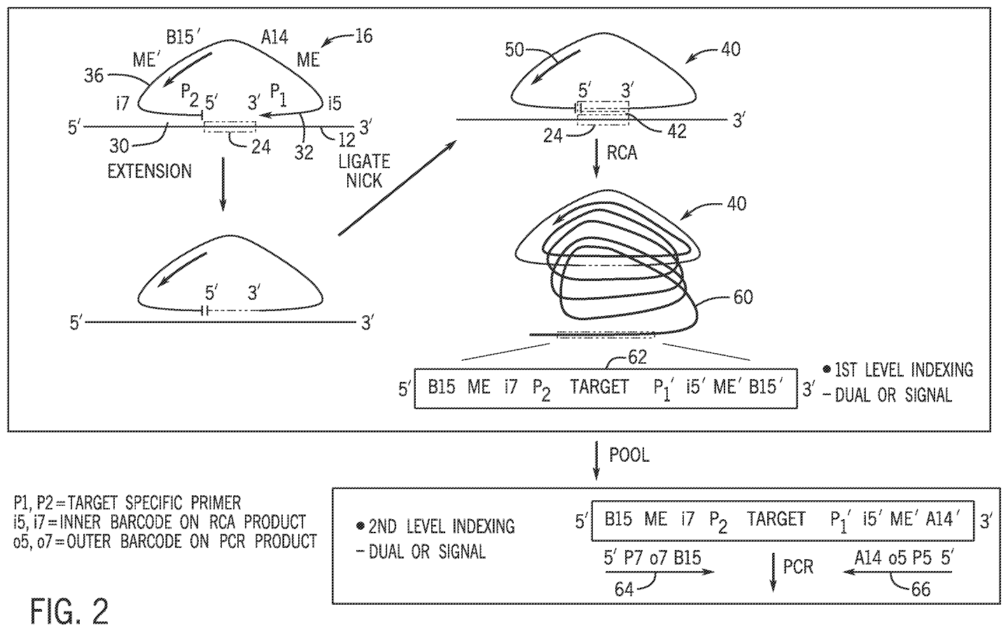

In the illustrated embodiment, different target binding sequences 20, 22 on the target nucleic acid flank different target regions 24 along the target nucleic acid 12. The technique includes providing oligonucleotides 16, e.g., single-stranded oligonucleotides 16, with two primers 30, 32 for different target regions 24 (e.g., target regions 24a, 24b, 24c, 24d) such that, when bound, an individual oligonucleotide 16 forms a looped structure around the target sequence 24 (see FIG. 2).

Each individual oligonucleotide 16 has a second target specific primer 30 that binds to a second target binding sequence 20 and a first target specific primer 32 that binds to a first target binding sequence 22. The second target specific primer 30 is 5′ of the first target specific primer 32 and is a reverse complement of the second target binding sequence 20. The first target specific primer 32 is a reverse complement of the first target binding sequence 22. To permit amplification of the target sequence 24 and, in some cases, preservation of variant information on the target nucleic acid 12, the oligonucleotide 16, before contact with the target nucleic acid and subsequent amplification, does not include a sequence complementary to the target sequence 24. In the illustrated embodiment, the second target specific primer 30 is at or near the 5′ end of the oligonucleotide and the first target specific primer 32 is at or near the 3′ end of the oligonucleotide 16.

To achieve coverage across the target nucleic acid 12, a plurality of oligonucleotides 16 may be provided, with distinguishable target specific primer sequences 30, 32 relative to one another and with binding specificity for different target binding sequences 20, 22 to amplify different target regions 24. That is, second target specific primer 30a has a different sequence than second target specific primer 30b, second target specific primer 30c, second target specific primer 30d, and so on. Further, first target specific primer 32a has a different sequence than first target specific primer 32b, first target specific primer 32c, first target specific primer 32d, and so on. Further, the target specific primer sequences 30, 32 may be different from one another to promote directional looped binding, as shown in FIG. 2.

While only a few oligonucleotides 16 are illustrated, it should be understood that a set of the oligonucleotides 16 may include two or more, five or more, 10 or more, 100 or more, or 1000 or more oligonucleotides 16. Different oligonucleotides 16 may be distinguishable from one another based on sequence. Further, the oligonucleotides 16 may be designed to achieve full coverage across the target nucleic acid 12, with target specific primer sequences 30, 32 designed for different target binding sequences 20, 22. In an embodiment, the oligonucleotides may be designed such that the target regions 24 represent only a portion of the target nucleic acid 12 in a targeted sequencing reaction. The oligonucleotides 16 may be provided as part of sample preparation reagents of a sequencing kit. In embodiments, the disclosed embodiments may include reaction mixtures or kits with 10-50, 10-100, or 10-500 oligonucleotides 16, each having primers 30, 32 that bind to different target binding sequences 20, 22 flanking a different target region 24. Further, reaction mixtures may include multiple copies of one or more individual oligonucleotides 16 with specificity for a particular target region 24 (e.g., target regions 24a, 24b, 24c, 24d). The number of different oligonucleotide target regions 24 may be selected based on desired assay characteristics.

In an embodiment, each individual oligonucleotide 16, in an embodiment, may be in a range of about 50-500 bases in length (e.g., 80-300 bases in length), and the target specific primer sequences 30, 32 may each individually be between 12-30 bases in length. While each oligonucleotide 16 includes a pair of target specific primer sequences 30, 32, the intervening region (e.g., 50-120 bases) between the target specific primer sequences 30, 32 may also include functional sequences, such as one or more barcodes or index sequences, sequencing primers, mosaic end sequences, etc. The length of the oligonucleotide 16 may vary according to the length of the target sequence 24, with longer oligonucleotides 16 being used with relatively longer target regions 24. In an embodiment, the target sequence 24 is between 1-2500 bases in length (e.g., 50-350 bases in length). In an embodiment, the target sequence 24 is between 100-200 bases in length, or about 150 bases in length, and suitable for short read sequencing.

FIG. 2 shows rolling circle amplification after binding of an individual oligonucleotide 16 to the target nucleic acid 12 as part of characterization of one or more target regions 24. At a first step, after combining the oligonucleotides 16 with the target nucleic acid 12 (see FIG. 1), the individual oligonucleotide 16 forms an open loop structure around an individual target sequence 24 on the target nucleic acid 12 via complementary binding of the of target specific primer sequences 30, 32. In the illustrated embodiment, the oligonucleotide 16 is provided with dual index sequences i5 and i7.

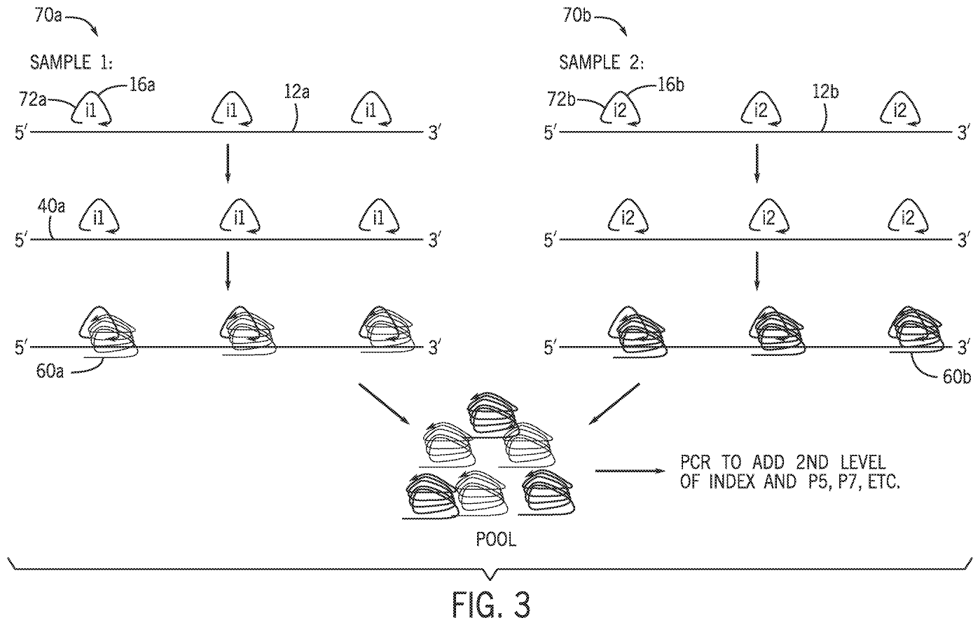

The intervening region 36 between the target specific primer sequences 30, 32 of the oligonucleotide 16 may include primer binding sequences, shown as B15′, A14, and mosaic end (ME) sequences by way of example. In an embodiment, at least one index sequence of the oligonucleotide 16 may be unique to the individual oligonucleotide 16 and distinguishable from indexes on the other oligonucleotides 16 in contact with the target nucleic acid 12. In a dual-indexing arrangement, the oligonucleotide may include two unique and distinguishable indexes. In an embodiment, the oligonucleotide 16 may include a sample barcode common to the reaction and indicative of the sample source in the reaction with the target nucleic acid 12. The primer binding sequence or sequences may be universal or common to the reaction with the target nucleic acid 12. In an embodiment, the primer binding sequence or sequences may be universal or common between different samples of a multiplexed reaction (see FIG. 3) to streamline protocol steps.

At a next step, the open loop structure is extended, via polymerase extension, at the 3′ end and towards the 5′ end and based on the target sequence 24 such that the added nucleotides form the reverse complements of the nucleotides in the target sequence 24. In an embodiment, the target nucleic acid is RNA, and the extension polymerase is an RT polymerase. In an embodiment, the target nucleic acid is DNA, and the extension polymerase is a DNA polymerase. The extension may be an isothermal reaction. The extended 3′ end is ligated to the 5′ end (e.g., via ligase). The 5′ end of the oligonucleotide 16 may be phosphorylated, before or after binding to the target nucleic acid 12, to promote ligation. Nick ligation closes the loop such that the oligonucleotide 16 forms a closed loop structure 40 that is modified via incorporation of the reverse complement 42 of the target sequence 24.

The closed loop structure 40 undergoes a rolling circle amplification reaction priming off of a sequence of the oligonucleotide 16 present in the closed loop structure. In an embodiment, the closed loop structure 40 may be heat-separated from the target nucleic acid 12 before initiating the rolling circle amplification via binding of a rolling circle amplification primer 50. However, in other reaction, such as one-pot reactions, the rolling circle amplification primer 50 may bind the oligonucleotide 16 at an earlier stage.

The rolling circle amplification primer 50 may be designed based on a common sequence between oligonucleotides 16 specific for different target regions 24 such that a single universal primer 50 amplifies all closed loop structures 40 in the reaction. Thus, in an embodiment, the primer 50 is specific for a sequence in the intervening region 36 and not the primers 30, 32. Rolling circle amplification generates a concatenated single-stranded nucleic acid 60 using a strand-displacing polymerase such as Phi29 polymerase, which has high processivity and strand displacing activity. The rolling circle amplification primer 50 may be 5-20 bases, for example. The rolling circle amplification reaction may be carried out using commercially available kits, for example the templiphi kit from Amersham Biosciences (GE Product number 25-6400-10) and with a custom primer 50 designed based on the sequence of the oligonucleotide 16.

It should be noted that the concatenated single-stranded nucleic acid 60 products of rolling circle amplification as disclosed herein are not circles, but are long strands of sequences where the circular material is copied multiple times in a linear strand. Each rolling circle amplification product is thus a long linear string containing concatemeric repeating copies of the circular sequence of the template, shown here as the closed loop 40. In an embodiment, the rolling circle amplification is run to an endpoint (e.g., depletion of dNTP reagents in the reaction mix). A repeating unit 62 of the concatenated single-stranded nucleic acid 60 includes the target sequence 24. Depending on the sequences present in the intervening region 36, functional sequences such as one or two index sequences, universal sequencing or primer binding sequences, enzyme binding sequences, etc., can be incorporated 5′ and/or 3′ of the target sequence 24 in the repeating unit 62. The concatenated single-stranded nucleic acid 60 may be pooled and subjected to PCR to add a second level of indexing that includes one or more additional index sequences and/or adapter sequences (e.g., P5 and P7, Illumina, Inc.) to generate fragments of a sequencing library in a standard format for particular sequencing platforms, such as Illumina sequencing platforms. Illustrated are primers 64, 66 that form a forward and reverse primer pair and that have additional 5′ sequences that are noncomplementary to the repeating unit 62 but that are incorporated into amplicons over the course of the amplification reaction.

In an embodiment, the primer binding sequences and index sequences of the intervening region 36 of the oligonucleotides 16 may be selected such that, when copied via rolling circle amplification, the complementary sequences incorporated into the repeating unit can go straight to sequencing to work with sequencing protocols as provided herein. Thus, the adapter sequences (e.g., P5 and P7, Illumina, Inc.) as well as any other relevant sequences can be directly incorporated into the repeating unit 62. In one example, different fragmentation sites may be present in the repeating unit to promote fragmentation of the concatenated single-stranded nucleic acid 60 for purposes of sequencing library preparation.

Thus, in the disclosed embodiments, a relatively low concentration target nucleic acid 12 can be provided as a starting material for characterization through the use of rolling circle amplification, which amplifies target sequences of interest while retaining variant information. Further, the disclosed synthetic oligonucleotides 16 can be used to generate size-controlled templates for the rolling circle amplification, which may be beneficial for generating fragments for characterization via short-read sequencing techniques that produce sequencing reads of about 150 bases and which are less costly than techniques using longer reads.

It should be understood that the oligonucleotides 16 may be in a single-stranded state prior to binding to the target nucleic acid 12. However, binding to the target nucleic acid 12 forms results in an at least partially double-stranded structure between the oligonucleotide 16 and the target nucleic acid 12. Further, the closed loop structure may be at least partially single-stranded during part of the disclosed protocol, but also forms at least partially double-stranded structures with the target nucleic acid 12, the rolling circle amplification primer 50, and during formation of the concatenated single-stranded nucleic acid 60.

FIG. 3 shows an example pooling step for a multiplexed reaction that occurs after generation of the concatenated single-stranded nucleic acid 60 in FIG. 2. Oligonucleotides 16a that bind to target nucleic acids 12a generated from a first sample 70a (Sample 1) are indexed. The indexing occurs via a sample 1-specific index 72a (shown as i1) via rolling circle amplification across a closed loop structure 40a generated from the oligonucleotide 16a bound to the target nucleic acid 12a. Oligonucleotides 16b that bind to target nucleic acids 12b generated from a second sample 70b (Sample 2) are indexed. The indexing occurs via a sample 2-specific index 72b (shown as i2) via rolling circle amplification across a closed loop structure 40b generated from the oligonucleotide 16b bound to the target nucleic acid 12b. Thus, the pooled concatenated single-stranded nucleic acids 60a, 60b are distinguishable and attributable to the or sample of origin based on the presence of an index sequence of Sample 1 or Sample 2. While the illustrated example shows two different samples in the multiplexed reaction, it should be understood that any number of samples may be present, each with different unique sample-specific indexes (i3, i4, i5, . . . in). The concatenated single-stranded nucleic acid 60 from different samples can be pooled after first-level indexing to undergo a PCR reaction for second-level indexing and/or adapter incorporation.

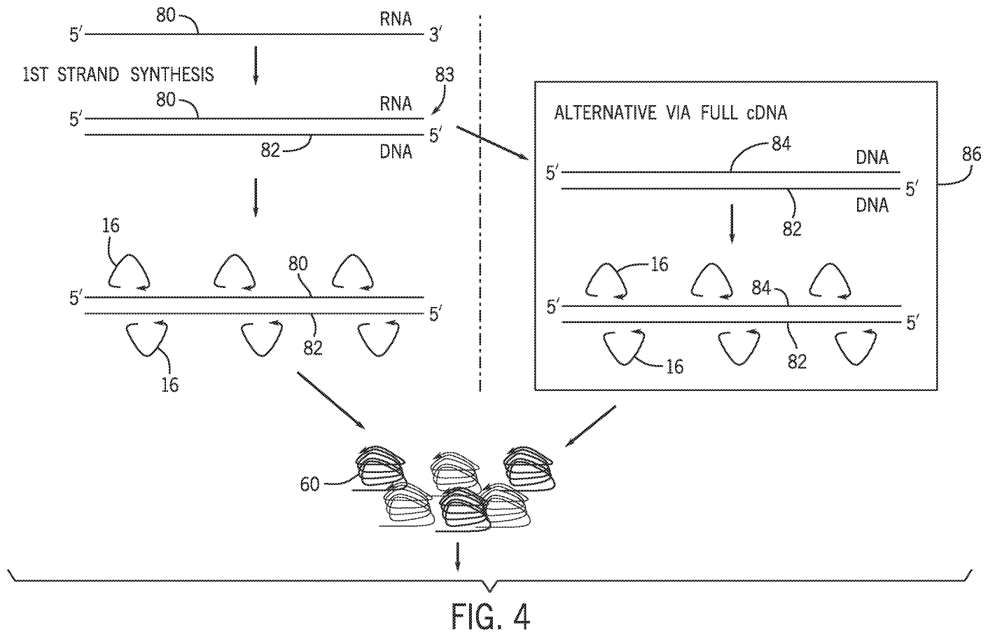

As provided herein, the target nucleic acid 12 may be a double-stranded or single-stranded RNA or DNA molecule. FIG. 4 shows a variation in which the target nucleic acid 12 includes cDNA to permit generation of both sense and antisense strand amplicon concatenated single-stranded nucleic acid 60. In one example, a template RNA strand 80 is used to generate a complementary cDNA strand 82 to form a double-stranded product 83 via reverse transcription. In another example, the double-stranded product 83 in turn is converted to a full double-stranded cDNA 86. Either, or both of these, in additional to the original single or double-stranded RNA, may be the target nucleic acid 12 in embodiments.

Sequences of strands 80, 82 of the double-stranded product 83 and/or the full cDNA 86 may be used to design oligonucleotides 16. In the depicted embodiments, the oligonucleotides 16 may be designed to bind at non-complementary target sequences on respective strands 80, 82 and/or 82, 84. However, it should be understood that the oligonucleotides may additionally or alternatively be designed to bind at complementary locations on the respective strands 80, 82 and/or 82, 84. The amplicon concatenated single-stranded nucleic acids 60 represent amplicons from two different strands, and can be indexed as disclosed herein and pooled for subsequent processing (additional indexing, sequencing). In embodiments, RCA amplicons can be separated from the template, e.g., via an exonuclease to digest away the template. The exonuclease may be RNA H in the case of an RNA template.

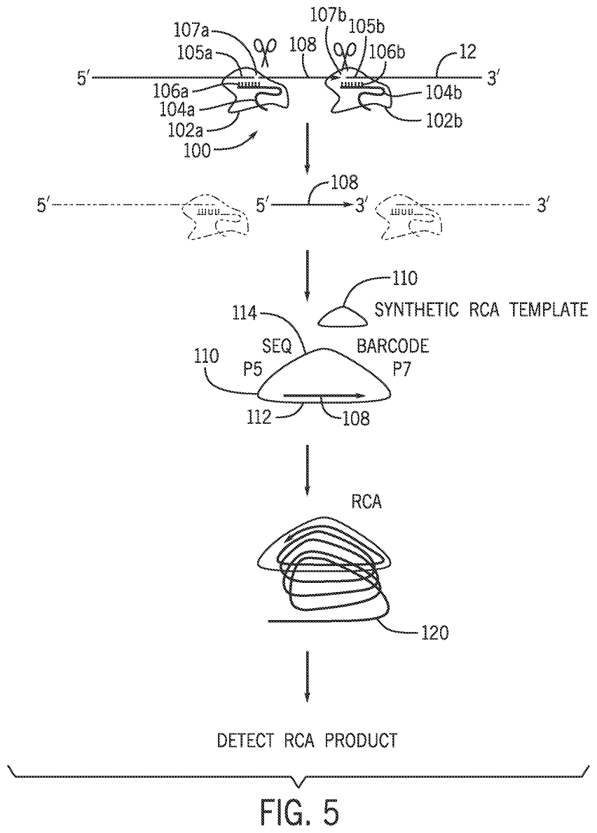

FIGS. 5-7 show examples of clustered regularly interspaced short palindromic repeats (CRISPR) and CRISPR-associated protein (Cas) interactions that additionally or alternatively may be used to generate primers used in conjunction with conventional and/or rolling circle amplification of a template molecule. As used provided herein, CRISPR-Cas refers to an enzyme system including a guide RNA sequence that contains a nucleotide sequence complementary or substantially complementary to a region of a target, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally occurring CRISPR-cas systems. CRISPR-Cas systems may contain engineered and/or mutated Cas proteins. CRISPR-Cas systems may contain engineered and/or programmed guide RNA. The guide RNA refers to a RNA containing a sequence that is complementary or substantially complementary to the target. A guide RNA may contain nucleotide sequences other than the region complementary or substantially complementary to a region of a target DNA sequence. A guide RNA may be a crRNA or a derivative thereof, e.g., a crRNA: tracrRNA chimera. In certain embodiments, the Cas protein is Cas9 protein, a Cas3 protein, or a Cas13 protein. For example, in embodiments in which the target nucleic acid is a ssRNA, the CRISPR-Cas includes a Cas13 protein that cleaves ssRNA.

Where a particular Cas protein functionality is specific to the form of the target nucleic acid 12 (e.g., single-stranded vs. doubles-stranded, RNA vs. DNA), it should be understood that the target nucleic acid 12 may undergo preprocessing steps to convert a single-stranded substrate to a double-stranded substrate, denature a single-stranded substrate, or synthesize a complementary DNA or RNA copy of one or both strands of the target nucleic acid 12. Accordingly, reaction mixes or kits as provided herein may include enzymes that are part of such pre-processing steps.

FIG. 5 shows an example CRISPR-Cas mediated reaction with a target nucleic acid 12. In the illustrated embodiment, the target nucleic acid 12 is a single-stranded RNA. However, the target nucleic acid 12 may be DNA and double or single stranded. The CRISPR-Cas system 100 includes a first Cas protein 102a and associated guide RNA 104a. The guide RNA 104a has a guide target-specific sequence 106a specific for a first target region 105a of the target nucleic acid 12. The CRISPR-Cas system 100 also include a second Cas protein 102b and associated guide RNA 104b. The guide RNA 104b has a guide target-specific sequence 106b specific for a second target region 105b of the target nucleic acid 12. Thus, the respective guide sequences 106a, 106b are specific for target-specific sequences 105, shown as spaced-apart target regions 105a, 105b on the target nucleic acid 12. In certain embodiments, the target regions 105a, 105b are on a same strand of the target nucleic acid 12 or may be on different strands of a double stranded target nucleic acid 12 where the Cas protein causes a double-stranded break. Cas-mediated cleavage at respective Cas cleavage sites 107a. 107b, within regions 105a, 105b cleaves out an intervening oligonucleotide 108 from between the 107a, 107b.

In embodiments, the guide target-specific sequences 106a, 106b are 17-20 bases in length. Thus, the size of the intervening oligonucleotide 108 may be dependent on the arrangement of the guide RNAs 104a, 104b on the target nucleic acid 12. In certain embodiments, the target nucleic acid 12 may be double-stranded, and the guide RNAs 104a, 104b may be designed to bind on separate strands while the Cas proteins 102a, 102b cause double-stranded breaks. In an embodiment, the guide sequences 106a, 106b bind to the same strand. In an embodiment, the 3′-bound guide target-specific sequence 106b on the same strand of the target nucleic acid 12 is shorter (e.g., 15-17 bases) than the more 5′ bound guide sequence 106a to permit a shorter oligonucleotide 108 to be released. In embodiments, the oligonucleotide is between 12-30 bases in length. The target regions 105a. 105b are spaced apart by approximately the length of the intervening oligonucleotide 108. However, in certain embodiment, the intervening oligonucleotide 108 includes some portions of the target regions 105a, 105b that are 3′ of the cleavage site for the first, or more 5′, Cas protein 102a and 5′ of the cleavage site for the second, or more 3′, Cas protein 102b. Thus, the base length between the 3′ end of the first target regions 105a and the 5′ end of the second target-specific sequence 105b may be less than the length of the intervening oligonucleotide 108.

The oligonucleotide 108, once released from the target nucleic acid 12, is free to serve as a rolling circle amplification primer for a circularized synthetic reporter template 110. The circularized synthetic reporter template 110 includes a sequence 112 complementary to the oligonucleotide 108. The circularized synthetic reporter template 110 may include additional functional sequences 114, such as adapter sequences, sequencing primer binding sites, one or more barcodes or indexes, etc. The generated concatenated single-stranded nucleic acid 120 can be detected/characterized according to techniques discussed herein.

The circularized synthetic reporter template 110 may, in embodiments, be a closed loop structure 40 generated from an oligonucleotide 16 as disclosed in FIGS. 1-4 and that is generated by binding of primers 30, 32 of the oligonucleotide 16 to the target binding sequences 20, 22 and subsequent closing of the loop. In embodiments, the circularized synthetic reporter template 110 and may be pre-formed and provided as a reagent to the reaction mixture. Accordingly, in embodiments, the reaction mixture reagents, which may be provided as a kit, may include the CRISPR-Cas system 100 with designed guide sequences 106a, 106b, the circularized synthetic reporter template 110, and reagents for rolling circle amplification, which may also include detection reagents.

In one embodiment, variants of a target nucleic acid (such as COVID-19 sequence variants or other pathogen variants) may be identified in a sample of the target nucleic acid 12 by providing different uniquely indexed circularized synthetic reporter templates 110 with primer binding sequences that represent respective complements of different variants of interest. The liberated oligonucleotide 108 will preferentially bind to the circularized synthetic reporter templates 110 that include the sequence 112 complementary to the oligonucleotide 108, including any variant present in the liberated oligonucleotide 108, and will have reduced binding to other circularized synthetic reporter templates 110 whose primer binding sequences do not complement the liberated oligonucleotide 108. Thus, at a detection stage, the index or indexes present in the generated concatenated single-stranded nucleic acid 120 can be subjected to short index reads, which are less costly that longer sequencing reads, to generate index sequence information which is associated with the particular associated variant of which the circularized synthetic reporter template 110 includes a complement. Accordingly, a reaction mixture or kit may include a plurality of circularized synthetic reporter templates 110 having subsets of templates 110 with respective different sequences 112 that represent different observed variants in the oligonucleotide 108. Further, while an example reaction was shown for the oligonucleotide 108, it should be understood that multiple sites of the target nucleic acid 12 may be included in a reaction to liberate multiple different oligonucleotides 108 in parallel at different locations. Thus, circularized synthetic reporter templates 110 provided as part of a reaction mixture may include different sequences 112 based on the sequences of the different liberated oligonucleotides 108.

While the embodiment of FIG. 5 shows a rolling circle amplification-based technique, the CRISPR-Cas system 100 can also be used to generate one or both primers of a conventional two primer amplification. In an embodiment, a second primer is provided as part of the reaction mix and works with the oligonucleotide 108 to amplify a synthetic template, which may be a linear synthetic template that generally may include features of the circularized synthetic reporter template 110 but arranged in a linear form.

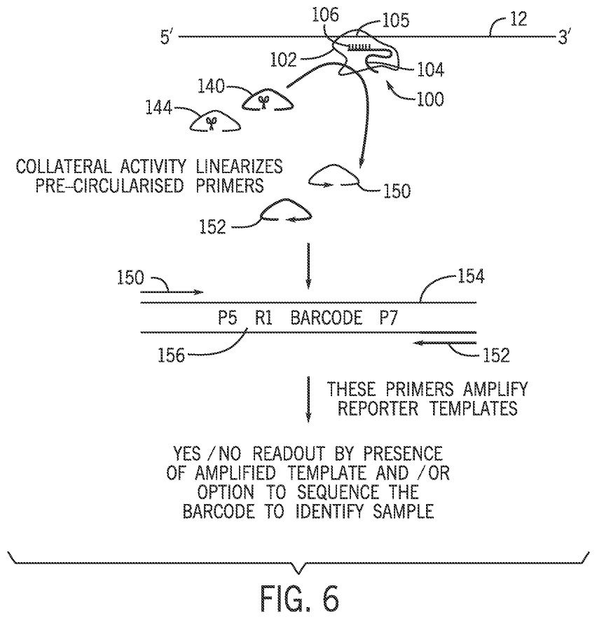

FIG. 6 shows an alternate CRISPR-Cas mediated reaction in which collateral cleavage activity of the CRISPR-Cas system 100 is harnessed to generate primers for amplification. The CRISPR-Cas system 100 is shown bound to a target nucleic acid 12, which includes a target region 105 having a sequence of interest. The system 100 is bound to the target nucleic acid 12 via the target-specific sequence 106 of the guide RNA 104 binding to the target region 105. Thus, binding of the system 100 via complementarity of the target-specific sequence 106 to the target region 105 is based on the presence of a particular sequence of the target region 105. The target-specific sequence 106 can be designed based on a particular sequence of interest. Collateral single-stranded cleaving enzyme activity of the Cas protein 102 (e.g., Cas13, Cas12a) that is activated by the binding can serve as part of a proxy indicator of the presence of the particular sequence of the target region 105. In the illustrated embodiment, collateral single-stranded cleaving activity is triggered by the specific binding to the target region 105 by the target-specific sequence 106 of the guide RNA. This cleaving can linearize single-stranded circular RNA templates 140, 144 to generate linearized primers 150, 152

Thus, in contrast to the example of FIG. 5 in which the primer is directly liberated from the target nucleic acid 12, e.g., viral RNA, FIG. 6 shows that the collateral activity of Cas can be used to liberate primers 150, 152 from circular templates 140, 144. The liberated primers may then participate in an amplification reaction on a reporter template 154. While the illustrated embodiment shows that the template 140, 144 represent a forward and reverse primer pair, only one of the forward or reverse primer may be provided as a circular template, with the other primer being spiked in already in linear form. The reporter template 154 may include functional sequences, such as a barcode or index to permit pooling of amplicons, and/or adapter sequences compatible with downstream sequencing reactions.

Accordingly, in embodiments, the reaction mixture reagents, which may be provided as a kit, may include the CRISPR-Cas system 100 with designed a guide sequence 106, one or more circular templates (e.g., circular templates 140, 144) representing one or both of a forward and reverse primer pair, a reporter template 154 to which the primer pair has specificity, and-in single primer embodiments, the other primer of the primer pair in linear form.

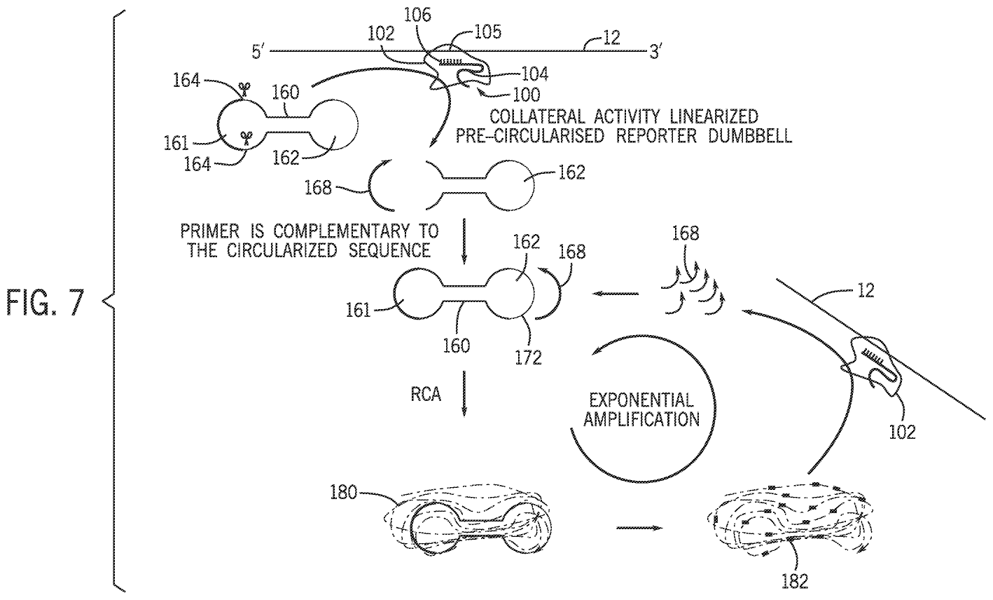

FIG. 7 is a schematic illustration of an embodiment in which the collateral activity of the Cas protein liberates a primer from a dumbbell. As in FIG. 6, the CRISPR-Cas system 100 is shown bound to a target nucleic acid 12, which includes a target region 105 having a sequence of interest. The system 100 is bound to the target nucleic acid 12 via the target-specific sequence 106 of the guide RNA 104 binding to the target region 105, which triggers collateral activity of the Cas protein 102. The collateral activity causes cleavage of a single-stranded dumbbell nucleic acid structure 160 that includes a first circular region 161 and a second circular region 162. Cleavage by the Cas protein 102 at spaced-apart cleavage sites 164 releases an oligonucleotide 168 that is a reverse complement of a sequence 172 in the second circular region 162.

Thus, the oligonucleotide 168 can serve as a primer for rolling circle amplification of another single-stranded dumbbell nucleic acid structure 160 that has not yet undergone cleavage at those sites and retains the first circular region 161 and a second circular region 162. A concatenated single-stranded nucleic acid amplification product 180 is generated, which may be detected via incorporation of detectable markers 182, in an embodiment. However, additional or alternative detection methods as provided herein are contemplated. Further, the concatenated single-stranded nucleic acid amplification product 180 is a target for the collateral Cas activity, which in turn generates more primers via cleavage. The primers in turn can generate more concatenated single-stranded nucleic acid amplification product 180 through rolling circle amplification of intact single-stranded dumbbell nucleic acid structures 160 in an exponential amplification. Accordingly, the ratio of single-stranded dumbbell nucleic acid structures 160 and CRISPR-Cas system 100 may be selected such that, even with very low target nucleic acid concentrations, the exponential amplification yields a robust detectable result of the concatenated single-stranded nucleic acid amplification product 180.

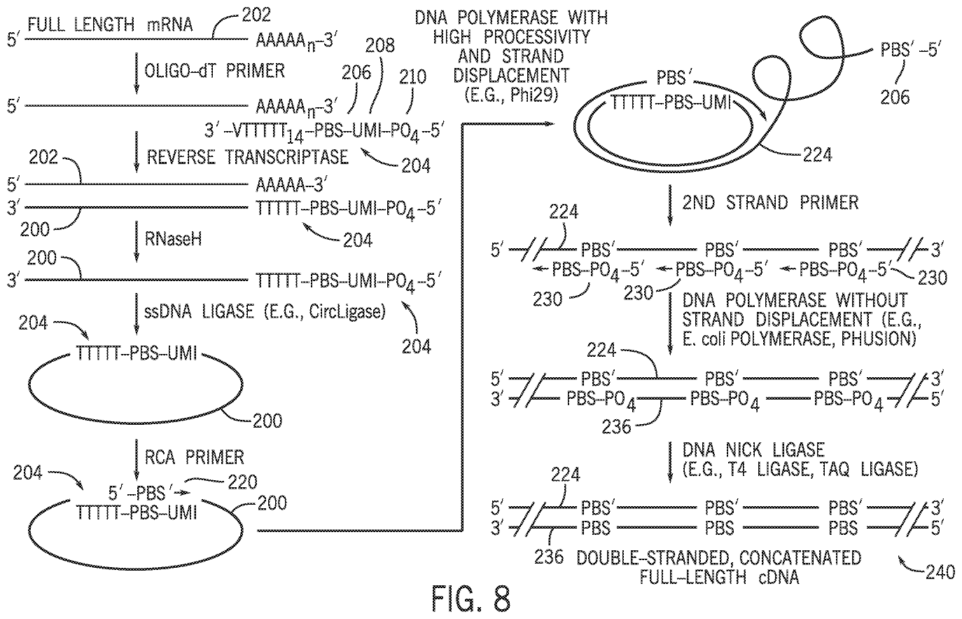

FIG. 8 shows an example rolling-circle amplification technique that permits exome sequencing via short reads but that retains phase information for variants to permit haplotyping. High-throughput, short-read DNA sequencing is a cost-effective way to sequence exomes with a low error rate. However, because the length of the reads is typically shorter than the full length of an mRNA, short-read technology is typically not able to produce an exome with the sequence of full-length mRNAs. This shortfall means that when processing short reads from cDNA, (1) mRNA isoforms (i.e., splice variants) cannot be fully analyzed (i.e., with the confidence that long-read technology can provide) and (2) variants present in the exome cannot be phased according to haplotype. Further, haplotype-phased variants in mRNA sequences are the haplotype-phased variants most likely to be interpretable and clinically actionable. Thus, retaining phase information for variants in exome sequencing would provide benefits over conventional short read sequencing techniques.

While certain sequencing techniques such as contiguity-preserving transposition (CPT) technology (i.e., Illumina spatial barcoding or sequence barcoding technology) retain phase information, full-length cDNA generated from mRNAs cannot be effectively sequenced using CPT. After tagmentation, linked reads generated from a cDNA typically only comprise approximately 10% of the full sequence. That is, CPT is inefficient at associating different parts of a cDNA with one another. The disclosed techniques involve generating a rolling circle-amplified cDNA substrate. The substrate is a concatenated nucleic acid generated from the cDNA that, when used in conjunction with CPT and short-read technology, allows generation of a full-length exome of a cDNA.

FIG. 8 shows a schematic overview of a protocol to concatenate full-length cDNA 200 to generate a long molecule of double-stranded DNA. First, to generate cDNA (i.e., a non-concatenated, single-copy of cDNA), full-length mRNA 202 is copied using reverse transcriptase and a 5′-phosphorylated oligo-dT primer 204. This DNA oligonucleotide primer 204 contains a primer binding site (PBS) 206 and, optionally, a unique molecular identifier (UMI) 208. The 5′ phosphorylated primer is capable of ligation at the 5′ phosphorylated end 210 in subsequent steps. In the illustrated embodiment, the oligo-dT primer has an optional 3′ V (A, G, or C).

After reverse transcription, the mRNA 202 is degraded using RNascH and the remaining cDNA 200 is circularized using a single-stranded DNA ligase (e.g., CircLigase). A DNA oligonucleotide primer 220 is then used to prime DNA synthesis at the PBS sequence 206. By using a DNA polymerase that is highly processive and capable of strand displacement (e.g., Phi29), concatenated copies 224 of the cDNA 200 are generated by rolling circle amplification (RCA). A 5′-phosphorylated DNA oligonucleotide primer 230 with the PBS sequence 206 is then used to prime synthesis of the complementary second strand. A DNA polymerase without strand displacement activity (e.g., E. coli ligase, Phusion) and a DNA nick ligase (e.g., T4 ligase, Taq ligase) are used to complete the complementary strand 236.

Using the double-stranded DNA product 240 as the assay substrate, Contiguity-Preserving Transposition (CPT) sequencing techniques can be used to generate a linked Illumina short-read library. CPT technology may be performed as generally disclosed in U.S. Pat. No. 10,557,133, incorporated herein by reference in its entirety for all purposes. Sequencing and analysis of this library yields a full-length exome. The concatenated nucleic acid can be used to generate a double-stranded DNA substrate with potentially greater than 100 copies of a cDNA concatenated end-to end (i.e., potentially >100 kb substrates). Because so many copies of the cDNA are now joined on a long stand of DNA, even when CPT technology only links a small fraction of the reads from this substrate strand, sequence redundancy in the concatenated substrate now enables splice-junctions and exome variants from the same haplotype to be effectively linked and analyzed.

In some embodiments, the disclosed techniques are used to generate a nucleic acid sequencing library or a DNA fragment library from the amplification products as provided herein. In one example, the library is generated from the nucleic acid by adding functional sequences, such as index sequences and primer binding sequences as part of the amplification techniques provided herein. Thus, the amplification products can be detected by sequencing the generated library in sequencing reactions to generate sequencing data. In an embodiment, the biological sample is a sample from a patient infected with a virus, e.g., COVID-19, or having a particular clinical condition, and the sequencing data includes a readout of variants detected in the sample using one or more of the disclosed amplification techniques. In one example, the amplification techniques amplify and sequence proxy templates, such as synthetic templates, rather than the sample itself. Thus, the readout may include yes/no indications for variants of interest. The sequencing data may include only shorter index/barcode reads, whereby the presence of a particular read linked to a particular first index ties the read to a particular patient in a multiplexed sample and the presence of a particular second index or UMI ties the read to a particular variant. Certain synthetic templates may only be amplified when upstream reactions tied to specific sequences in the target nucleic acid liberate a primer to permit amplification of a synthetic templates. In additional examples, the synthetic templates may be complementary to the liberated primer and, therefore, the synthetic template sequences may provide variant information.

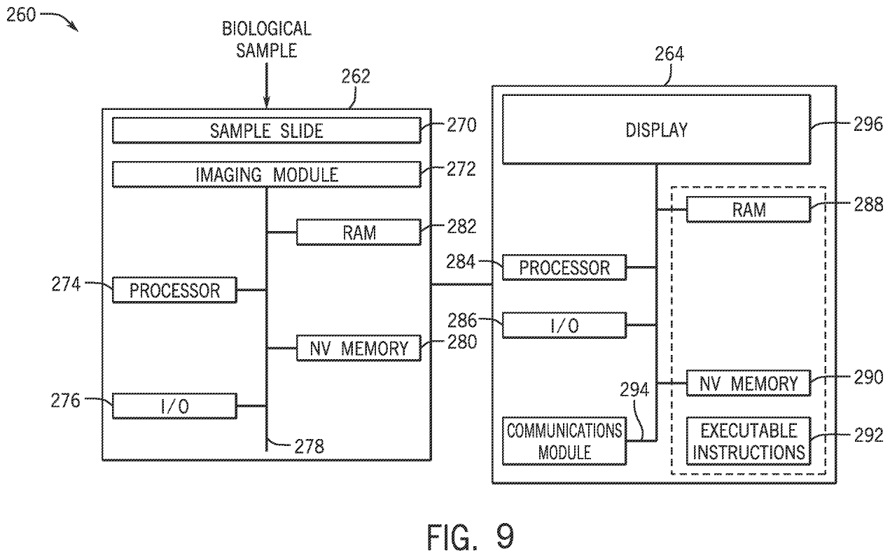

FIG. 9 is a schematic diagram of a sequencing device 260 that may be used in conjunction with the disclosed embodiments for acquiring sequencing data from nucleic acids (e.g., sequencing reads, read 1, read 2, index reads, index read 1, index read 2, multi-sample sequencing data) as provided herein. The sequence device 260 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 260. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties. Some embodiments can utilize nanopore sequencing, whereby sample nucleic acid strands, or nucleotides exonucleolytically removed from sample nucleic acids, pass through a nanopore. As the sample nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing device 260 may be an iSeq from Illumina (La Jolla, CA). In other embodiment, the sequencing device 260 may be configured to operate using a CMOS sensor with nanowells fabricated over photodiodes such that DNA deposition is aligned one-to-one with each photodiode.

The sequencing device 260 may be a “one-channel” detection device, in which only two of four nucleotides are labeled and detectable for any given image. For example, thymine may have a permanent fluorescent label, while adenine uses the same fluorescent label in a detachable form. Guanine may be permanently dark, and cytosine may be initially dark but capable of having a label added during the cycle. Accordingly, each cycle may involve an initial image and a second image in which dye is cleaved from any adenines and added to any cytosines such that only thymine and adenine are detectable in the initial image but only thymine and cytosine are detectable in the second image. Any base that is dark through both images in guanine and any base that is detectable through both images is thymine. A base that is detectable in the first image but not the second is adenine, and a base that is not detectable in the first image but detectable in the second image is cytosine. By combining the information from the initial image and the second image, all four bases are able to be discriminated using one channel. In other embodiments, the sequencing device 260 may be a “two-channel” detection device

In the depicted embodiment, the sequencing device 260 includes a separate sample substrate 262, e.g., a flow cell or sequencing cartridge, and an associated computer 264. However, as noted, these may be implemented as a single device. In the depicted embodiment, the biological sample may be loaded into substrate 262 that is imaged to generate sequence data. For example, reagents that interact with the biological sample fluoresce at particular wavelengths in response to an excitation beam generated by an imaging module 272 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imaging module 272, which may be a camera or other optical detector.

The imaging module detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imaging module 272 may be under processor control, e.g., via a processor 274, and may also include I/O controls 276, an internal bus 278, non-volatile memory 280, RAM 282 and any other memory structure such that the memory is capable of storing executable instructions, and other suitable hardware components that may be similar to those described with regard to FIG. 10. Further, the associated computer 264 may also include a processor 184, I/O controls 286, a communications module 284, and a memory architecture including RAM 288 and non-volatile memory 290, such that the memory architecture is capable of storing executable instructions 292. The hardware components may be linked by an internal bus 294, which may also link to the display 296. In embodiments in which the sequencing device 260 is implemented as an all-in-one device, certain redundant hardware elements may be eliminated.

The processor 284 may be programmed to assign individual sequencing reads to a sample based on the associated index sequence or sequences according to the techniques provided herein. In particular embodiments, based on the image data acquired by the imaging module 272, the sequencing device 260 may be configured to generate sequencing data that includes sequence reads for individual clusters, with each sequence read being associated with a particular location on the substrate 270. Each sequence read may be from a fragment containing an insert. The sequencing data includes base calls for each base of a sequencing read. Further, based on the image data, even for sequencing reads that are performed in series, the individual reads may be linked to the same location via the image data and, therefore, to the same template strand. In this manner, index sequencing reads may be associated with a sequencing read of an insert sequence before being assigned to a sample of origin. The processor 284 may also be programmed to perform downstream analysis on the sequences corresponding to the inserts for a particular sample subsequent to assignment of sequencing reads to the sample.

While the disclosed amplification product may be detected by generating sequencing data as provided herein, e.g., via the sequencing device 260, additional detection methods are also contemplated. Target sequences or amplification products can be detected in a detection method of the disclosed embodiments using rolling circle amplification (RCA) or conventional amplification. This can be accomplished in a variety of ways; for example, the primer, e.g., the rolling circle amplification primer, can be labeled or the polymerase can incorporate labeled nucleotides and labeled product detected by a capture probe in a detection array. Rolling-circle amplification can be carried out under conditions such as those generally described in Baner et al. (1998) Nuc. Acids Res. 26:5073-5078; Barany, F. (1991) Proc. Natl. Acad. Sci. USA 88:189-193; and Lizardi et al. (1998) Nat Genet. 19:225-232. In addition the rolling circle amplification products to be easily detected by hybridization to probes in a solid-phase format (e.g. an array of beads). An additional advantage of the RCA is that it provides the capability of multiplex analysis so that large numbers of sequences can be analyzed in parallel. In additional, hybridization-based detection on an array and/or quantitative PCR-based detection techniques are also contemplated.

The disclosed techniques may be used to characterize a target nucleic acid (e.g., target nucleic acid 12). “Target nucleic acid” or sample nucleic acid can be derived from any in vivo or in vitro source, including from one or multiple cells, tissues, organs, or organisms, whether living or dead, or from any biological or environmental source (e.g., water, air, soil). For example, in some embodiments, the sample nucleic acid comprises or consists of eukaryotic and/or prokaryotic dsDNA that originates or that is derived from humans, animals, plants, fungi, (e.g., molds or yeasts), bacteria, viruses, viroids, mycoplasma, or other microorganisms. In some embodiments, the sample nucleic acid comprises or consists of genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In some embodiments, the sample nucleic acid comprises multiple dsDNA molecules in or prepared from nucleic acid molecules (e.g., multiple dsDNA molecules in or prepared from genomic DNA or cDNA prepared from RNA in or from a biological (e.g., cell, tissue, organ, organism) or environmental (e.g., water, air, soil, saliva, sputum, urine, feces) source. In some embodiments, the sample nucleic acid is from an in vitro source. For example, in some embodiments, the sample nucleic acid comprises or consists of dsDNA that is prepared in vitro from single-stranded DNA (ssDNA) or from single-stranded or double-stranded RNA (e.g., using methods that are well-known in the art, such as primer extension using a suitable DNA-dependent and/or RNA-dependent DNA polymerase (reverse transcriptase). In some embodiments, the sample nucleic acid comprises or consists of dsDNA that is prepared from all or a portion of one or more double-stranded or single-stranded DNA or RNA molecules using any methods known in the art, including methods for: DNA or RNA amplification (e.g., PCR or reverse-transcriptase-PCR (RT-PCR), transcription-mediated amplification methods, with amplification of all or a portion of one or more nucleic acid molecules); molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray.

The disclosed concatenated nucleic acids, CRISPR-modified sequences, and/or primer arrangements may include non-naturally occurring nucleic acid sequences or synthetic nucleic acid sequences.

This written description uses examples as part of the disclosure to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A nucleic acid composition, comprising:
a first oligonucleotide comprising a first 5′ primer sequence, a first 3′ primer sequence, and a first intervening region disposed between the first 5′ primer sequence and the first 3′ primer sequence;
a second oligonucleotide comprising a second 5′ primer sequence, a second 3′ primer sequence and a second intervening region disposed between the second 5′ primer sequence and the second 3′ primer sequence; and
a target nucleic acid, wherein the first 5′ primer sequence and the first 3′ primer sequence are complementary to first regions flanking a first target sequence of the target nucleic acid and wherein the second 5′ primer sequence and the second 3′ primer sequence are complementary to second regions flanking a second target sequence of the target nucleic acid such that the first oligonucleotide, when bound to the target nucleic acid, forms a first looped structure about the first target sequence and the second oligonucleotide, when bound to the target nucleic acid, forms a second looped structure around the second target sequence.

2. The composition of claim 1, wherein the first target sequence and the second target sequence are between 50-350 bases in length.

3. The composition of claim 1, wherein the target nucleic acid is a single-stranded RNA.

4. The composition of claim 1, comprising a polymerase that is capable of extending the first looped structure between the first 5′ primer sequence and the first 3′ primer sequence and extending the second looped structure between the second 5′ primer sequence and the second 3′ primer sequence.

5. The composition of claim 4, wherein the polymerase is an RT polymerase.

6. The composition of claim 4, comprising a ligase that is capable of closing the first looped structure by ligating an extended first 3′ end to the first 5′ primer sequence and the second looped structure by ligating an extended second 3′ end to the second 5′ primer sequence.

7. The composition of claim 1, comprising a rolling circle amplification primer that is specific for a common sequence in the first intervening region and the second intervening region.

8. The composition of claim 1, comprising a first single-stranded concatenated nucleic acid comprising repeating units, the repeating units comprising the first target sequence and complements of the first 5′ primer sequence, the first 3′ primer sequence, and the first intervening region.

9. (canceled)

10. The composition of claim 1, wherein the first intervening region and the second intervening region have a same sequence.

11. (canceled)

12. The composition of claim 1, wherein the first 5′ primer sequence binds the target nucleic acid 5′ of the first target sequence and wherein the first 3′ primer sequence binds 3′ of the first target sequence.

13. The composition of claim 1, wherein the second 5′ primer sequence binds the target nucleic acid 5′ of the second target sequence and wherein the second 3′ primer sequence binds 3′ of the second target sequence.

14. (canceled)

15. A method for amplifying a target sequence, comprising:
contacting a target nucleic acid with an oligonucleotide such that the oligonucleotide binds to spaced-apart target binding sequences on the nucleic acid to form a looped structure about a target sequence of the target nucleic acid;
extending a 3′ end of the oligonucleotide towards a 5′ end and across the target sequence;
ligating the extended 3′ end to the 5′ end of the oligonucleotide to form a closed loop; and
using the closed loop as a template for rolling circle amplification to generate a concatenated single-stranded nucleic acid.

16. The method of claim 15, comprising detecting or sequencing the concatenated single-stranded nucleic acid to detect the target sequence.

17. (canceled)

18. The method of claim 15, comprising pooling the concatenated single-stranded nucleic acid with other concatenated single-stranded nucleic acids, the concatenated single-stranded nucleic acid comprising a unique index sequence not present in the other concatenated single-stranded nucleic acids and amplifying the pooled concatenated single-stranded nucleic acid to introduce one or more additional index sequences.

19. (canceled)

20. A method for detecting a target nucleic acid comprising:
providing a system having a first clustered regularly interspaced short palindromic repeats (CRISPR) guide RNA and a first CRISPR-associated (Cas) protein and a second CRISPR guide RNA and a second Cas protein, wherein the first guide RNA contains a target-specific nucleotide region complementary to a first region of a target nucleic acid and the second guide RNA contains a target target-specific nucleotide region complementary to a second region of a target nucleic acid spaced apart from the first region;
contacting the target nucleic acid with the system to form a complex to cleave within the first region and the second region to release an oligonucleotide comprising intervening nucleotides between the first region and the second region;
annealing the oligonucleotide to a template; and
amplifying the template using the annealed oligonucleotide as a primer.

21. The method of claim 20, comprising detecting or sequencing the amplified template to detect the oligonucleotide.

22.-24. (canceled)

25. A method for detecting a target nucleic acid comprising:
providing a system having a clustered regularly interspaced short palindromic repeats (CRISPR) guide RNA and a CRISPR-associated (Cas) protein, wherein the guide RNA contains a target-specific nucleotide region complementary to a region of a target nucleic acid;
providing a plurality of circularized oligonucleotides;
contacting the target nucleic acid with the system to form a complex;
linearizing the plurality of circularized oligonucleotides to generate primers using the Cas protein in the complex;
annealing one or more of the primers to a template; and
amplifying the template using the one or more of the primers annealed to the template.

26.-28. (canceled)

29. (canceled)

30. The method of claim 25, wherein the plurality of circularized oligonucleotides comprise a dumbbell structure have a first circular region linked to a second circular region, and wherein linearizing the plurality of circularized oligonucleotides to generate primers comprises linearizing only the first circular region and not the second circularized portion, and wherein the template comprises the second circular region.

31. (canceled)

32. A method for amplifying an mRNA target nucleic acid comprising:
providing a primer for a reverse transcriptase reaction, the primer comprising a primer binding sequence for rolling circle amplification and a phosphorylated 5′ end;
annealing the primer to an mRNA;
extending the primer using reverse transcriptase to generate a cDNA comprising the primer;
ligating the phosphorylated 5′ end of the primer in the cDNA to a 3′ end to circularize the cDNA;
annealing a rolling circle amplification primer to the primer binding sequence of the circularized cDNA; and
amplifying the circularized cDNA using the rolling circle amplification primer annealed to the circularized cDNA to generate a concatenated single-stranded nucleic acid.

33. The method of claim 32, comprising annealing second strand primers to repeating units of the concatenated single-stranded nucleic acid and extending the annealed second strand primers to synthesize a second strand of the concatenated single-stranded nucleic acid.

34.-36. (canceled)
